(19) United States
(12) Patent Application Publication
(10) Pub. No.: US 2026/0306701 A1
(43) Pub. Date: Oct. 8, 2026

(54) METHODS AND COMPOSITIONS FOR WOUND CARE

(71) Applicant: NOVABONE PRODUCTS, LLC, Alachua, FL (US)

(72) Inventors: Ashley M. COMPAAN, Alachua, FL (US); Daniel NUNEZ, Alachua, FL (US); Douglas RODRIGUEZ, Alachua, FL (US)

(21) Appl. No.: 19/490,249

(22) PCT Filed: Jun. 5, 2024
(86) PCT No.: PCT/US2024/032601

§ 371 Date: Dec. 4, 2025

Related U.S. Application Data
(60) Provisional application No. 63/506,199, filed on Jun. 5, 2023.

(51) Int. Cl. A61L 26/00

(52) U.S. Cl. CPC A61L 26/0033; A61L 26/0004; A61L 26/0066; A61L 26/0085; A61L 26/0095; A61L 2400/06

(57) ABSTRACT

Disclosed are compositions comprising collagen and bioactive glass. In some aspects, the compositions further comprise a cross-linking agent. Disclosed are methods of making a sheet-like composition comprising preparing a collagen slurry; lyophilizing, cross-linking, and adding bioactive glass. Disclosed are methods of making a flowable composition comprising preparing a collagen slurry; lyophilizing, and adding bioactive glass.

### CROSS-REFERENCE TO RELATED APPLICATIONS

This application claims the benefit of U.S. Application No. 63/506,199, filed on Jun. 5, 2023, the contents of which are incorporated herein by reference in their entirety.

### BACKGROUND

The majority of chronic and deep tunneling wounds are treated by repeated saline lavage followed by a temporary dressing. The clinical result is generally limited to ineffective tissue repair leading to a continued chronic open wound, infection and/or scarring.

There is a continued need for a safe and effective wound care treatment.

### BRIEF SUMMARY

Disclosed are compositions comprising collagen and bioactive glass. In some aspects, the compositions further comprise a cross-linking agent.

Disclosed are compositions comprising about 60-95%/wt collagen; about 0-4%/wt of a cross-linking agent; and about 1-40%/wt bioactive glass.

Disclosed are delivery devices filled with one or more of the disclosed compositions

Disclosed are methods of making a sheet-like composition comprising preparing a collagen slurry; lyophilizing, cross-linking, and adding bioactive glass.

Disclosed are methods of making a flowable composition comprising preparing a collagen slurry; lyophilizing, and adding bioactive glass.

Disclosed are methods of making a sheet-like composition comprising preparing a collagen slurry; lyophilizing, cross-linking, adding bioactive glass, rehydrating, adding more collagen slurry, lyophilizing, cross-linking, and sterilizing.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1C show schematic diagrams of examples of the disclosed compositions in a sheet configuration comprising different size bioactive glass.

FIGS. 2A-2D show schematic diagrams of examples of the disclosed compositions in a sheet configuration comprising different size bioactive glass and further comprising a collagen bilayer.

FIG. 3 shows cross sections of skin defects treated with a disclosed composition, showing progress of wound healing at 7 days (scale bars: 100 μm).

FIG. 4 shows images of different bacterial species which were eradicated after exposure to bioactive glass.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Headings are provided for convenience only and are not to be construed to limit the invention in any manner. Embodiments illustrated under any heading or in any portion of the disclosure may be combined with embodiments illustrated under the same or any other heading or other portion of the disclosure.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a “, “an”, and “the” include plural reference unless the context clearly dictates otherwise. Thus, for example, reference to “a cross-linking agent” includes a plurality of such cross-linking agents, reference to “the bioactive glass” is a reference to one or more bioactive glasses and equivalents thereof known to those skilled in the art, and so forth.

As used herein, the term “subject” or “patient” can be used interchangeably and refer to any organism to which a composition of the invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates, and the like.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Compositions

Disclosed are compositions comprising collagen and bioactive glass. In some aspects, the compositions can be in a sheet-like configuration. In some aspects, the compositions can be in the form of a flowable material.

Disclosed are compositions comprising about 60-95%/wt collagen and about 1-40%/wt bioactive glass, wherein the collagen is cross-linked. Disclosed are compositions comprising about 3-60%/wt collagen and about 40-97%/wt bioactive glass, wherein the collagen is cross-linked. Disclosed are compositions comprising about 5-97%/wt collagen and about 1-99%/wt bioactive glass, wherein the collagen is cross-linked.

Disclosed are compositions comprising 78% collagen, 20% bioactive glass, and 2% cross-linking agent.

Disclosed are compositions comprising about 60-95%/wt collagen; about 0-4%/wt of a cross-linking agent; and about 1-40% bioactive glass. Disclosed are compositions comprising about 70-90%/wt collagen; about 1.5-2.5%/wt of a cross-linking agent; and about 10-40% bioactive glass. Disclosed are compositions comprising about 70-85%/wt collagen; about 1.5-2.5%/wt of a cross-linking agent; and about 15-40% bioactive glass. In some aspects, disclosed are compositions having about 20%/wt bioactive glass, 78%/wt collagen, and 2%/wt cross-linking agent (e.g., citric acid).

In some aspects of the disclosed compositions, the cross-linking agent is optional. For example, disclosed are compositions comprising about 3-60%/wt collagen and about 40-97% bioactive glass.

In some aspects of the disclosed compositions, the collagen is cross-linked due to the presence of a cross-linking agent. Examples of cross-linking agents include, but are not limited to citric acid, glycine, lysine, glutamic acid, serine, trilysine, glutaraldehyde, transglutaminase, ethylene oxide, phosphate (cross linking aid), tris, carbodiimide, EDC-NHS.

In some aspects, the disclosed compositions can be lyophilized or freeze-dried.

In some aspects, the disclosed compositions have a density of about 25+/−20 mg/cc. For example, in some aspects, the disclosed compositions have a density of 18.8-31.8 mg/cc.

In some aspects, the disclosed compositions have a pH of about 7-10. In some aspects, the disclosed compositions have a pH of about 8-10. In some aspects, the higher pH can be due to the basic bioactive glass despite the acidic collagen matrix.

In some aspects, the disclosed compositions have a particle size of about 10-1000 μm. In some aspects, the disclosed compositions have a particle size of about 10-100 μm.

In some aspects, the disclosed compositions have a mass of about 0.2-20 grams. In some aspects, the disclosed compositions can be in a sheet-like configuration. In some aspects a 10 in x 20 in sheet-like configuration can have a mass of about 16 g.

In some aspects, the disclosed compositions can be in a flowable form. In some aspects, the disclosed compositions can have a volume of about 1-1500 cc.

In some aspects, the disclosed compositions can have a density of about 5-100 mg/cc. In some aspects, the composition can have a density of about 25+/−20 mg/cc. In some aspects, the disclosed compositions can be sterilized.

In some aspects, the disclosed compositions can further comprise a therapeutic agent. In some aspects, the therapeutic agent can be a signaling protein, a glycosaminoglycan, a growth factor, an antimicrobial agent, an anti-inflammatory, an analgesic, an antibiotic, or a combination thereof.

In some aspects, the disclosed compositions do not comprise chondroitin sulfate.

In some aspects, the compositions disclosed herein can be stable for at least three weeks. In some aspects, the lyophilized composition can be stable for at least three months. In some aspects, the disclosed compositions can be stable for 1, 2, 3, 4, 5, 6, 7, 8, 9, 10, 11, 12, 24, 36, 48, or 60 months.

### 1. Collagen

Disclosed are compositions comprising collagen and bioactive glass. For example, disclosed are compositions comprising about 60-95%/wt collagen; about 0-4%/wt of a cross-linking agent; and about 1-40%/wt bioactive glass. In some aspects, the collagen can be marine (e.g. fish), bovine, porcine, ovine, vegan, equine, mammalian, jellyfish, avian, reptilian, amphibian, bioengineered, or recombinant collagen. In some aspects, the collagen can be from any area of a human, cow, pig, sheep, rodent, or horse. For example, the collagen can be skin-derived, tendon-derived or hide-derived.

In some aspects, the collagen is purified collagen, wherein it has been purified from any of the sources described herein. In some aspects, the collagen has negligible amounts of non-collagen components derived from the collagen source. For example, the collagen has negligible amounts of cells, fat, growth factors, or other proteins.

In some aspects, the collagen is synthetic or recombinant. In some aspects, synthetic collagen retains the structural characteristics. In some aspects, the synthetic or recombinant collagen can be any material that can form a resorbable sponge.

In some aspects, the collagen of the disclosed compositions is cross-linked.

In some aspects, the disclosed compositions comprise about 3-50%, 3-60%, 60-95%, or 60-99%/wt collagen. In some aspects, the disclosed compositions comprise about 78% collagen.

In some aspects, the collagen of the disclosed compositions can be acid buffer treated or alkaline buffer treated.

In some aspects, there can be a first layer of collagen and a second layer of collagen in the disclosed compositions.

In some aspects, a collagen slurry can be poured into molds having specific shapes for the disclosed compositions.

i. Bilayer

In some aspects, the disclosed compositions comprise a bilayer of collagen meaning they have two layers of collagen. Thus, disclosed are compositions comprising a first layer of collagen mixed with bioactive glass and a second layer of collagen. In some aspects, the second layer of collagen does not comprise bioactive glass. In some aspects, the layer comprising collagen only is the first layer and the layer comprising collagen slurry mixed with bioactive glass is considered the second layer.

In some aspects, the second layer of collagen, or the layer with collagen only, is cross-linked. Thus, in some aspects, the second layer of collagen comprises the same cross-linking agent as the first layer of collagen, or the layer with collagen mixed with bioactive glass.

In some aspects, the bilayer is for additional functionality. In some aspects, if the bilayer is on a side of the composition that can contact a wound then the bilayer provides an additional denser layer of collagen to draw matrix metalloproteinases/collagenases away from the wound thus facilitating the initial wound healing process. In some aspects, if the bilayer is on a side opposite of that which contacts a wound then the bilayer provides a means to slow evaporation of wound fluids since the bilayer is denser. In some aspects, the bilayer provides additional means to suture the composition in-place.

In some aspects, the concentration of collagen in the first layer is the same concentration of the collagen in the second layer. In some aspects, the concentration of collagen in the first layer is higher or lower than the concentration of the collagen in the second layer. In some aspects, the concentration of collagen in the second layer is higher or lower than the concentration of the collagen in the first layer.

In some aspects, the density of collagen in the first layer is 10-200 mg/cc. In some aspects, the density of collagen in the second layer is 10-200 mg/cc. In some aspects, the density of collagen in the first layer is the same density of the collagen in the second layer. In some aspects, the density of collagen in the first layer is higher or lower than the density of the collagen in the second layer. In some aspects, the density of collagen in the second layer is higher or lower than the density of the collagen in the first layer.

### 2. Cross-Linking Agent

In some aspects, the disclosed compositions comprise a cross-linking agent. In some aspects, the cross-linking agent is citric acid, glycine, lysine, glutamic acid, serine, trilysine, glutaraldehyde, transglutaminase, ethylene oxide, phosphate (cross linking aid), tris, carbodiimide, or EDC-NHS. In some aspects, the cross-linking agent is citric acid, wherein the citric acid is in a final concentration of about 1-3%. In some aspects, the cross-linking agent is citric acid, wherein the citric acid is in a final concentration of about 2%/wt. In some aspects, the concentration of cross-linking agent present in the composition can be about 0-15%. In some aspects, the cross-linking agent can be used at a concentration of 0.05%.

In some aspects, disclosed are compositions comprising collagen and bioactive glass, wherein the collagen is cross-linked but no cross-linking agent is present. For example, cross-linking of the collagen can occur using physicothermal methods, including, but not limited to, UV cross-linking, gamma irradiation, e-beam irradiation, and dehydrothermal treatment, and therefore no cross-linking agent is present in the composition.

### 3. Bioactive Glass

In some aspects, the disclosed compositions can comprise bioactive glass.

In some aspects, the bioactive glass is 45S5, 58S, S53P4, or a Borate-formulation. In some aspects, the bioactive glass comprises 12-45% CaO, 0-65% SiO2, 0-30% Na₂O, 2-17% P₂O₅, 0-7% MgO, 0-1% CaF₂, 0-60% B₂O₃, and 0-15% K₂O by mass percent.

In some aspects, the bioactive glass can be formed via melt or sol-gel glass processing methods. In some aspects, bioactive glass can have dense or porous morphologies. In some aspects, porous morphology results in higher surface area, leading to faster rates of dissolution.

In some aspects, the bioactive glass has a particle size of about 1-1000 μm. For example, the composition can have a mean particle size of about 50. In some aspects, the bioactive glass is in the form of particles having a particle size of about 1-600 μm.

In some aspects, the disclosed composition comprise a non-porous bioactive glass. In some aspects, the disclosed composition comprise a porous bioactive glass. In some aspects, the bioactive glass has a porosity of up to 90%. In some aspects, the bioactive glass has pores ranging from about 1 to about 1000 microns. In some aspects, the bioactive glass has an average pore size of 100 microns plus or minus 50 microns, 200 microns plus or minus 50 microns, 300 microns plus or minus 50 microns, 400 microns plus or minus 50 microns, 500 microns plus or minus 50 microns, 600 microns plus or minus 50 microns, 700 microns plus or minus 50 microns.

In some aspects, the bioactive glass is in the form of irregular granules. In some aspects, the bioactive glass is in the form of approximately spherical particles. In some aspects, the bioactive glass is in the form of fibers.

In some aspects, a smaller size range of bioactive glass can be used for a homogenously dispersed monolayer composition. In some aspects, the bioactive glass is naturally buoyant and therefore no settling occurs via gravity.

In some aspects, a larger size range of bioactive glass can be used for a composition that is functionally graded through-the-thickness. In some aspects, larger bioactive glass can settle near bottom of the composition.

In some aspects, a combination of smaller and larger size ranges of bioactive glass are present in the disclosed compositions, with smaller particles dispersed throughout the composition and large particles that are pre-dominantly on one side due to settling.

In some aspects, the composition comprises about 3-60%/wt collagen and about 40-97%/wt bioactive glass. In some aspects, the composition comprises about 3-50%/wt collagen and about 50-90%/wt bioactive glass. In some aspects, the composition comprises about 5-97%/wt collagen and about 1-99%/wt bioactive glass. In some aspects, the composition comprises about 78%/wt collagen and about 20%/wt bioactive glass.

In some aspects, the bioactive glass described throughout can be substituted for one or more of the following: Beta-tricalcium phosphate (B-TCP), hydroxyapatite (HA), carbonated hydroxyapatite, calcium sulfate, calcium phosphate, calcium apatite, aragonite, or any mixture of these with each other or with bioactive glass. Thus, any of disclosed compositions or methods of making or using the disclosed compositions can have any of these substitutes instead of bioactive glass. For example, in some aspects, the composition comprises about 3-60%/wt collagen and about 40-97%/wt B-TCP. In some aspects, the composition comprises about 3-50%/wt collagen and about 50-90%/wt B-TCP. In some aspects, the composition comprises about 5-97%/wt collagen and about 1-99%/wt B-TCP.

### 4. Sheet-Like Configuration

In some aspects, the disclosed compositions can be in the form of a sheet-like configuration. In some aspects, the sheet-like configuration can make it easier to apply to a wound bed, wherein the composition is pre-hydrated or in a dry form (e.g. dehydrated or lyophilized).

In some aspects, the disclosed compositions can be in a malleable or flowable form.

In some aspects, the disclosed compositions can be molded into any desired shape without loss of homogeneity. In some aspects, the disclosed compositions can be in a form similar to soft tissue (e.g. malleable and less rigid than bone).

In some aspects, a sheet configuration can range from 0.5 in² to 100 in², including circular, square, and rectangular sheets. In some aspects, the sheet-like configurations can be any shape.

In some aspects, the disclosed compositions can have a thickness of 1-10 mm.

In some aspects, the disclosed compositions can be in a sheet like configuration and can further comprise a layer of collagen deposited on one side of the composition. For example, a collagen slurry comprising bioactive glass that has been lyophilized and rehydrated can then be coated in a layer of collagen slurry and applied to the disclosed compositions. In some aspects, this results in a composition having a bilayer of collagen.

In some aspects, the disclosed compositions comprise a bilayer of collagen meaning they have two layers of collagen. Thus, disclosed are compositions comprising a first layer of collagen mixed with bioactive glass and a second layer of collagen. In some aspects, the second layer of collagen does not comprise bioactive glass. In some aspects, a first layer of collagen does not comprise bioactive glass and a second layer of collagen is mixed with bioactive glass.

In some aspects, the second layer of collagen, or the layer with collagen only, is cross-linked. Thus, in some aspects, the second layer of collagen comprises the same cross-linking agent as the first layer of collagen, or the layer with collagen mixed with bioactive glass.

In some aspects, the bilayer is for additional functionality. In some aspects, if the bilayer is on a side of the composition that can contact a wound then the bilayer provides an additional denser layer of collagen to draw matrix metalloproteinases/collagenases away from the wound thus facilitating the initial wound healing process. In some aspects, if the bilayer is on a side opposite of that which contacts a wound then the bilayer provides a means to slow evaporation of wound fluids since the bilayer is denser. In some aspects, the bilayer provides additional means to suture the composition in-place.

In some aspects, the concentration of collagen in the first layer is the same concentration of the collagen in the second layer. In some aspects, the concentration of collagen in the first layer is higher or lower than the concentration of the collagen in the second layer. In some aspects, the concentration of collagen in the second layer is higher or lower than the concentration of the collagen in the first layer.

In some aspects, the density of collagen in the first layer is 10-200 mg/cc. In some aspects, the density of collagen in the second layer is 10-200 mg/cc. In some aspects, the density of collagen in the first layer is the same density of the collagen in the second layer. In some aspects, the density of collagen in the first layer is higher or lower than the density of the collagen in the second layer. In some aspects, the density of collagen in the second layer is higher or lower than the density of the collagen in the first layer

### 5. Flowable Material

In some aspects, the disclosed compositions can be in the form of a flowable material (e.g. a flowable form). In some aspects, the flowable material makes it easier to apply to certain wound sites, such as tunneled wounds.

In some aspects, the flowable material can be present in a delivery device, such as a syringe. In some aspects, the syringe is a 1 cc; 2.5 cc; or 5 cc syringe.

In this configuration (i.e. the form of a flowable material), the loose material can be packaged in a syringe or other delivery device. In some aspects, the flowable form can be lyophilized or freeze-dried and can be hydrated upon use by mixing of the composition with a hydration fluid. Examples of hydration fluids include, but are not limited to water, saline, pharmaceuticals (e.g., analgesics, growth factors, antimicrobials, anti-inflammatories, etc.), plasma, platelet rich plasma, serum, blood, cell suspensions, or bone marrow aspirate.

In some aspects, the composition and hydration fluid can be separately packaged in different syringes and the composition can be hydrated by mixing the composition and hydration fluid between two syringes for a given number of cycles.

### C. Delivery Device

Disclosed are delivery devices filled with one or more of the disclosed compositions. Disclosed are delivery devices filled with one or more of the disclosed compositions wherein the composition are in the form of a flowable material.

In some aspects, a delivery device can be a syringe. Thus, in some aspects, disclosed are syringes filled with the product produced by the methods of producing a flowable material disclosed herein. In some aspects, the syringe is a 1 cc; 2.5 cc; 5 cc or 10 cc syringe

In some aspects, a delivery device can be the NovaBone MIS cartridge delivery system as described in U.S. Pat. No. 9,199,032, hereby incorporated by reference in its entirety herein.

In some aspects, disclosed is a delivery device wherein the force required to eject the moldable wound matrix composition (e.g. collagen compositions disclosed herein) from the applicator is less than 20 lbs.

### D. Methods of Treating

Disclosed are methods of managing a wound or tissue defect comprising administering one or more of the disclosed compositions to the wound or tissue defect. In some aspects, managing a wound or soft tissue defect comprises repairing or treating the wound or soft tissue defect.

Disclosed are methods of treating a wound or tissue defect comprising administering one or more of the disclosed compositions to the wound or tissue defect.

In some aspects, the wound can be a partial thickness or full thickness wound. For example, a wound can be selected from the group consisting of a laceration, a scrape, an abrasion, a thermal or chemical burn, an incision, a puncture, a wound caused by a projectile, a chronic wound, an acute wound, an external wound, an internal wound, a congenital wound, an ulcer, and combinations thereof. In some aspects, a wound or tissue defect can be in connection with surgery. For example, a surgery can be selected from the group consisting of a tendon surgery, a ligament surgery, a bone surgery, a spine surgery, a laminectomy, a knee surgery, a shoulder surgery, a hand surgery, an elbow surgery, a toe surgery, a foot surgery, an ankle surgery, a laprascopic surgery, an endoscopic surgery, robotic surgery, an open abdominal surgery, or combinations thereof.

In some aspects, the wound is specifically referred to as a skin wound. In some aspects, a skin wound can be a partial or full thickness wound such as a pressure ulcer, venous ulcer, diabetic ulcer, chronic vascular ulcer, tunneled/undermined wound, surgical wound (donor site/graft, post-Moh's surgery, post-laser surgery, podiatric, wound dehiscence), trauma wound (abrasion, laceration, second-degree burn, or skin tear), or draining wound.

In some aspects, administering can mean placing the composition on the wound or tissue defect. In some aspects, administering can mean ejecting, from a delivery device, the flowable composition onto a wound or tissue defect.

### E. Methods of Making

Disclosed are methods of making a composition in a sheet-like configuration or flowable material.

### 1. Sheet-Like Configuration

Disclosed are methods of making a composition comprising preparing a collagen slurry; lyophilizing, cross-linking, and adding bioactive glass. Examples of each of these steps is described in more detail below and the order of each of these steps can be changed. For example, cross-linking can be performed before or after lyophilization and rehydrating can be performed before or after adding the bioactive glass.

In some aspects, several optional steps can also be performed, including, but not limited to, rehydrating, milling, coating with a second collagen layer, perforation.

In some aspects, disclosed are methods of making a composition comprising preparing a collagen slurry; lyophilizing the collagen slurry to create a lyophilized collagen sample; cross-linking the collagen slurry before or after lyophilization; adding bioactive glass to a cross-linked lyophilized collagen sample; and optionally, rehydrating the cross-linked lyophilized collagen sample to create a rehydrated cross-linked collagen sample; optionally, coating the rehydrated cross-linked collagen sample with a collagen slurry; optionally, lyophilizing the collagen slurry-coated sample to create a lyophilized collagen-bioactive glass sample; and optionally, cross-linking the collagen-bioactive glass sample.

i. Prepare Collagen Slurry

The disclosed methods can comprise the step of preparing a collagen slurry. In some aspects, the collagen slurry comprises collagen and a buffer.

In some aspects, preparing the collage slurry comprises obtaining a collagen sample. In some aspects, the collagen can be any of those disclosed herein, for example, human, bovine, porcine, ovine or equine collagen.

In some aspects, the collagen slurry is a 0.9-1.25% (m/v) slurry. The composition, when lyophilized, is based on dry mass and the ~99% water in the slurry is removed during lyophilization to leave only the collagen (and crosslinker, if applicable) component in the final composition resulting in a composition having 60-95%/wt collagen.

In some aspects, the collagen slurry can comprise a higher concentration which will create a denser end product. In some aspects, a higher concentration can be >1.1% m/v. In some aspects, a higher concentration can be up to about 2.5% m/v in the slurry. In some aspects, an increased density can lead to slower resorption of the entire composition. In some aspects, a higher concentration would result in a dry density on the order of 30-200 mg/cc in the sample.

In some aspects, the method comprises adjusting the pH to about 7-10. In some aspects, the collagen slurry can further comprise a pH adjuster. Thus, in some aspects, the collagen used to prepare the slurry can be either acid buffer treated or alkaline buffer treated. In some aspects, preparing the collagen slurry comprises adding a pH adjuster, such as, but not limited to, HCl, citric acid, acetic acid, NaOH, or ammonia hydroxide.

In some aspects, instead of adding the pH adjuster to the slurry, the collagen itself can have its pH adjusted prior to making the slurry. For example, collagen can be immersed in an acid or alkaline bath and agitated for a given period of time in order to adjust the pH. In some aspects, the collagen is then rinsed with water to remove excess salts from the previous pH adjusting bath

In some aspects, preparing the collagen slurry comprises homogenization. In some aspects, homogenizing can be performed while keeping the temperature below 20° C. results in more uniformity. In some aspects, an ice bath, cooling blanket, or temperature controlled bowl can be used to keep temperatures low. In some aspects, the homogenized slurry can be degassed, for example, via centrifugation or vacuum method. In some aspects, the homogenized slurry can be gently mixed following degassing since gravitational separation can occur.

In some aspects, preparing the collagen slurry can include adding a cross-linking agent to the collagen and buffer. In some aspects, although a cross-linking agent is present in the collagen slurry, the step of cross-linking may not occur until after lyophilization. In some aspects, cross-linking can occur prior to the first lyophilization.

In some aspects, after preparing the collagen slurry, the collagen slurry can be poured into a container or mold. In some aspects, the container or mold can be used to prepare the collagen slurry in a specific shape and/or size. Thus, in some aspects, after the step of preparing a collagen slurry, the step of pouring the collagen slurry into a mold can be performed.

In some aspects, the buffer can be a pharmaceutically acceptable carrier. Thus, in some aspects, the disclosed compositions can comprise a pharmaceutically acceptable carrier. For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art.

In some aspects, the composition can comprise a biocompatible buffer, thus, the pharmaceutically acceptable carrier can be a biocompatible buffer. For example, the composition can comprise citrate, acetate, phosphate, formate, tris, MES, and/or succinate buffer systems. The slurry can also comprise additional acids, bases, or salts for pH or handling adjustment, such as sodium hydroxide, potassium hydroxide, hydrochloric acid, acetic acid, sodium carbonate, sodium bicarbonate, sodium sulfate, sulfuric acid, sodium chloride, potassium chloride, sodium acetate, potassium sulfate, potassium carbonate, or potassium bicarbonate.

ii. First Lyophilization Step

The disclosed methods of making the disclosed compositions can comprise a first lyophilization step. In some aspects, the first lyophilization step comprises lyophilizing the collagen slurry to create a lyophilized collagen sample. In some aspects, if the first lyophilization step is performed after the cross-linking then the first lyophilization step comprises lyophilizing the cross-linked collagen slurry to create a cross-linked lyophilized collagen sample.

In some aspects, the collagen slurry is dispensed into trays before the first lyophilization step. In some aspects, there can be about 1 L of collagen slurry/tray. In some aspects, there can be about 500 to 3000 mL of collagen slurry/tray. In some aspects, the trays can be about 11.5×23.5 in. In some aspects, the trays can be about 29.2×59.7 cm.

In some aspects, after freezing, the collagen slurry is then dried. In some aspects, the drying can be performed at the same temperature as the freezing or can be performed at a temperature 1°, 5°, 10°, 15°, 20°, 25°, 30°, 35°, 40°, 45°, 50°, 55°, 60°, 65°, 70°, 75°, 80°, 85°, or 90° C. higher than the temperature of the freezing step.

In some aspects, the drying step after freezing can be carried out for less than 10 hours. In some aspects, the drying step can be carried out for 10, 12, 14, 16, 18, 20, or 24 hours. In some aspects, the drying step can be carried out for 24, 48 or 72 hours. In some aspects, the drying step can be carried out for 12-144 hours.

In some aspects, these steps of lyophilization create a lyophilized collagen sample.

iii. Perforation

In some aspects of the methods of making the disclosed compositions, a step of perforation can be performed. In some aspects, the lyophilized collagen sample can be perforated.

In some aspects, perforation can be performed by using a pinned roller tool. In some aspects, perforation can be performed using any tool that allows for perforating or puncturing the composition.

iv. Milling

In some aspects of the methods of making the disclosed compositions further comprise a step of milling the lyophilized collagen sample. In some aspects, milling includes powdering, comminuting, dividing, or morselizing the lyophilized collagen sample.

In some aspects, milling can be performed after cross-linking. Thus, in some aspects, the disclosed methods comprise the steps of lyophilizing, milling and then cross-linking; lyophilizing, cross-linking, and then milling; and cross-linking, lyophilizing, and then milling.

v. First Cross-Linking

In some aspects of the methods of making the disclosed compositions can include the step of cross-linking before or after a first lyophilization.

The disclosed methods of making the disclosed compositions can comprise the step of crosslinking the lyophilized collagen sample to form a cross-linked lyophilized collagen sample.

The disclosed methods of making the disclosed compositions can comprise the step of crosslinking the collagen slurry to form a cross-linked collagen sample.

In some aspects, a crosslinking agent is mixed in, or added to, the collagen slurry prior to lyophilization which helps stabilize the collagen as a lyophilized sheet. In some aspects, a cross-linking agent can be added after lyophilization. In some aspects, heat treatment under a vacuum can be used to crosslink the collagen. In some aspects, regardless of when a cross-linking agent is added to the collagen slurry, the cross-linking may not occur until activation of the cross-linking agent. Thus, even if the cross-linking agent is added while preparing the collagen slurry, the step of cross-linking can occur after lyophilization upon activation of the cross-linking agent.

In some aspects, the cross-linking agent can be any of those disclosed herein. In some aspects, the cross-linking agent used during the cross-linking step can be, but is not limited to, citric acid, glycine, lysine, glutamic acid, serine, trilysine, glutaraldehyde, transglutaminase, ethylene oxide, phosphate (cross linking aid), tris, carbodiimide, or EDC-NHS. In some aspects, the cross-linking agent is citric acid, wherein the citric acid is in a final concentration of about 1-3%/wt. In some aspects, the cross-linking agent is citric acid, wherein the citric acid is in a final concentration of about 2%/wt.

In some aspects, the cross-linking step can occur using methods, such as, but not limited to, UV, ethylene oxide, glutaraldehyde vapor, enzymatic crosslinking, dehydrothermal treatment, physicochemical crosslinking, and physicothermal crosslinking Thus, any known method of cross-linking can be used in the disclosed methods.

In some aspects, cross-linking agent concentrations can range from about 0.05% to 5% (on a dry weight basis) of the final composition.

In some aspects, the cross-linking agent provides the handling and resorption characteristics for the final composition. In some aspects, cross-linking can be determined by testing how strong the composition is and by testing how the composition degrades in a collagenase solution.

vi. Adding Bioactive Glass

The disclosed methods of making the disclosed compositions can comprise the step of adding bioactive glass. In some aspects, the disclosed methods of making the disclosed compositions can comprise the step of adding bioactive glass to the cross-linked lyophilized collagen sample. Thus, in some aspects, the bioactive glass can be added prior to lyophilization or after lyophilization.

In some aspects, adding the bioactive glass can be performed before or after the step of rehydrating. In some aspects, adding the bioactive glass can be performed after perforating the lyophilized collagen sample. Thus, in some aspects, the bioactive glass can be dispensed onto a perforated surface of the lyophilized collagen sample.

In some aspects, the bioactive glass is added to a single side, two sides, or all sides of the rehydrated collagen sample.

In some aspects, bioactive glass can be added dry or pre-mixed with a liquid, such as water or a buffer. In some aspects, 0.3-10 g of bioactive glass powder is added per sheet (or per collagen sample).

In some aspects, adding bioactive glass to the cross-linked lyophilized collagen sample comprises spreading or sprinkling the bioactive glass for even coverage.

In some aspects, the bioactive glass can be any of those disclosed herein. In some aspects, the bioactive glass is 45S5, 58S, S53P4, or a Borate-formulation. In some aspects, the bioactive glass comprises 12-45% CaO, 0-65% SiO2, 0-30% Na₂O, 2-17% P₂O₅, 0-7% MgO, 0-1% CaF₂, 0-60% B₂O₃, and 0-15% K₂O by mass percent.

In some aspects, a smaller size range of bioactive glass can be used for a homogenously dispersed monolayer composition. In some aspects, the bioactive glass is naturally buoyant and therefore no settling occurs via gravity.

In some aspects, a larger size range of bioactive glass can be used for a composition that is functionally graded through-the-thickness. In some aspects, larger bioactive glass can settle near bottom before the composition is frozen in the lyophilization process.

In some aspects, a combination of smaller and larger size ranges of bioactive glass can be added to create a composition with smaller particles dispersed throughout the composition and large particles that are pre-dominantly on one side due to settling.

In some aspects, the bioactive glass has a particle size of about 10-1000 μm. For example, the composition can have a mean particle size of about 50 μm. In some aspects, the bioactive glass is in the form of particles having a particle size of about 1-600 μm.

In some aspects, the disclosed compositions made by the disclosed methods comprise a non-porous bioactive glass. In some aspects, the disclosed composition comprise a porous bioactive glass. In some aspects, the bioactive glass has a porosity of up to 90%. In some aspects, the bioactive glass has pores ranging from about 1 to about 1000 microns. In some aspects, the bioactive glass has an average pore size of 100 microns plus or minus 50 microns, 200 microns plus or minus 50 microns, 300 microns plus or minus 50 microns, 400 microns plus or minus 50 microns, 500 microns plus or minus 50 microns, 600 microns plus or minus 50 microns, 700 microns plus or minus 50 microns.

In some aspects, the bioactive glass is in the form of irregular granules. In some aspects, the bioactive glass is in the form of approximately spherical particles. In some aspects, the bioactive glass is in the form of fibers.

In some aspects, the bioactive glass is dispersed uniformly throughout the composition. In some aspects, the bioactive glass is not dispersed uniformly throughout the composition.

vii. Rehydrating

The disclosed methods of making the disclosed compositions can comprise the step of rehydrating. Thus, in some aspects, the disclosed methods of making the disclosed compositions comprise the step of rehydrating the cross-linked lyophilized collagen sample to create a rehydrated cross-linked collagen sample.

In some aspects, rehydrating the lyophilized collagen sample comprises contacting the lyophilized collagen sample with a pharmaceutically acceptable buffer, including, but not limited to, water or phosphate buffered saline (PBS).

In some aspects, rehydrating comprises placing the lyophilized collagen sample in a container comprising a buffer, such as water. In some aspects, the lyophilized collagen sample can be in contact with (e.g. soaking in) a buffer for about 1 min, 2 min, 3 min, 4 min, 5 min, 6 min, 7 min, 8 min, 9 min, 10 min or longer. In some aspects, the lyophilized collagen sample can be in contact with (e.g. soaking in) a buffer for at least 1-60 min.

In some aspects, rehydrating comprises using an amount of buffer sufficient to cover the lyophilized collagen sample. In some aspects, at least 50 mL, 100 mL, 150 mL, 200 mL, 250 mL, 300 mL, 350 mL, 400 mL, 450 mL, 500 mL, 550 mL, 600 mL, 650 mL, 700 mL, 750 mL, 800 mL, 850 mL, 900 mL, 950 mL, or 1000 mL can be used.

viii. Coating Sample with Collagen Slurry

In some aspects, the disclosed compositions made by the methods of making the disclosed compositions comprise a collagen bilayer. In some aspects, after rehydrating, the composition can be coated with a collagen slurry. In some aspects, this can be considered a bilayer because the initial collagen slurry is being coated with another layer of collagen slurry.

In some aspects, the collagen slurry coated on the rehydrated composition can be the same collagen slurry prepared and used in the early steps of the disclosed methods. In some aspects, a new collagen slurry is prepared prior to coating the collagen slurry.

In some aspects, the disclosed methods of making the disclosed compositions comprise the addition, or coating, of a thin, dense layer of a collagen slurry on one side of the composition using the steps described herein through the rehydration step. In some aspects, the composition with the bilayer of collagen is then air dried to create a thin, dense film of collagen.

In some aspects, the coating of a collagen slurry comprises coating with a collagen slurry that does not contain bioactive glass. In some aspects, the coating of a collagen slurry comprises coating with a collagen slurry that comprises bioactive glass. In some aspects, a low collagen concentration slurry is used in the coating of the collagen slurry. In some aspects, a low collagen concentration slurry comprises <1% m/v.

In some aspects, coating the composition with a collagen slurry comprises painting, spraying, or pouring the collagen slurry on the top or bottom surface of the composition.

In some aspects, both sides of the rehydrated collagen sample can be coated with the collagen slurry. In some aspects, a single side (e.g. top or bottom) can be coated with the collagen slurry.

ix. Second Lyophilization Step

In some aspects, the disclosed methods of making the disclosed compositions can comprise a second lyophilization step. In some aspects, the second lyophilization can be performed in the same manner as the first lyophilization.

In some aspects, after freezing, the collagen slurry is then dried. In some aspects, the drying can be performed at the same temperature as the freezing or can be performed at a temperature 1°, 5°, 10°, 15°, 20°, 25°, 30°, 35°, 40°, 45°, or 50° C. higher than the temperature of the freezing step.

In some aspects, the drying step after freezing can be carried out for less than 10 hours. In some aspects, the drying step can be carried out for 10, 12, 14, 16, 18, 20, or 24 hours. In some aspects, the drying step can be carried out for 24, 48 or 72 hours. In some aspects, the drying step can be carried out for 12-144 hours.

In some aspects, these steps of a second lyophilization create a lyophilized collagen sample coated with collagen.

x. Second Cross-Linking

In some aspects, the disclosed methods of making the disclosed compositions can comprise a second cross-linking step.

In some aspects, the second cross-linking step allows for cross-linking of the collagen slurry that was coated onto the rehydrated composition.

In some aspects, the second crosslinking can occur prior to the second lyophilization. In some aspects, the second cross-linking can occur after the second lyophilization.

In some aspects, the cross-linking agent can be any of those disclosed herein. In some aspects, the cross-linking agent used during the cross-linking step can be, but is not limited to, citric acid, glycine, lysine, glutamic acid, serine, trilysine, glutaraldehyde, transglutaminase, ethylene oxide, phosphate (cross linking aid), tris, carbodiimide, or EDC-NHS. In some aspects, the cross-linking agent is citric acid, wherein the citric acid is in a final concentration of about 1-3%/wt. In some aspects, the cross-linking agent is citric acid, wherein the citric acid is in a final concentration of about 2%/wt.

In some aspects, the second cross-linking step can occur using methods, such as, but not limited to, UV, ethylene oxide, glutaraldehyde vapor, enzymatic crosslinking, dehydrothermal treatment, gamma irradiation, physicochemical crosslinking, and physicothermal crosslinking In some aspects, any known method of cross-linking can be used in the disclosed methods.

In some aspects, cross-linking agent concentrations can range from about 0.05% to 5% (on a dry weight basis) of the final composition.

In some aspects, the same cross-linking agent used in the first cross-linking can be used in the second cross-linking. In some aspects, a different cross-linking agent from what was used in the first cross-linking can be used in the second cross-linking.

xi. Sterilizing

In some aspects, the disclosed methods of making the disclosed compositions further comprise the step of sterilizing the compositions. In some aspects, sterilizing can be performed as the final step of the method of producing the disclosed compositions. Thus, in some aspects, sterilizing can be performed after the first lyophilization, after cross-linking, after adding the bioactive glass, after rehydration, after the coating, after the second lyophilization, or after cutting to the desired size.

In some aspects, any method of sterilization can be performed, for example, but not limited to, ethylene oxide, gamma irradiation, or ebeam irradiation.

xii. Cutting Compositions

In some aspects, the disclosed compositions can be cut to a desired size. Cutting a composition to a desired size can occur prior to freezing the collagen sample or after lyophilization or after cross-linking.

In some aspects, the disclosed compositions can be cut to a size of 2 cm×2 cm; 3 cm×3 cm; 5 cm×5 cm; 7.5 cm×7.5 cm; 10 cm×12 cm; 10 cm×25 cm; 17.5 cm×17.5 cm; or 20 cm×30 cm.

In some aspect, the disclosed compositions can be cut to rectangles, squares, or circles. In some aspects, the dimensions can range from 2.5 cm to 30 cm. In some aspects, the compositions can range from 0.5 in² to 100 in², including circular, square, and rectangular sheets.

### 2. Flowable Material

Disclosed are methods of making a composition that is a flowable material.

Disclosed are methods of making a composition comprising preparing a collagen slurry; lyophilizing, and adding bioactive glass. Each of these steps is described in more detail below and the order of each of these steps can be changed.

i. Prepare Collagen Slurry

The disclosed methods of making a composition that is a flowable material can comprise the step of preparing a collagen slurry. In some aspects, the collagen slurry comprises collagen and a buffer. In some aspects, the collagen is a lyophilized collagen that needs to be rehydrated prior to using in the collagen slurry.

In some aspects, preparing the collage slurry comprises obtaining a collagen sample. In some aspects, the collagen can be any of those disclosed herein, for example, human, bovine, porcine, ovine or equine collagen.

In some aspects, the collagen slurry is a 0.9-1.25% (m/v) slurry.

In some aspects, the collagen slurry can comprise a higher concentration which will create a denser end product. In some aspects, a higher concentration can be >1.1% m/v. In some aspects, a higher concentration can be up to about 2.5% m/v in the slurry. In some aspects, an increased density can lead to slower resorption of the entire composition. In some aspects, a higher concentration would result in a dry density on the order of 30-200 mg/cc in the sample. In some aspects, an increased density can lead to slower resorption of the entire composition.

In some aspects, the methods of making a composition that is a flowable material comprises adjusting the pH to about 7-10. In some aspects, the collagen slurry can further comprise a pH adjuster. Thus, in some aspects, collagen is either acid buffer treated or alkaline buffer treated. In some aspects, preparing the collagen slurry comprises adding a pH adjuster, such as, but not limited to, HCl, citric acid, acetic acid, NaOH, or ammonia hydroxide. In some aspects, an alkaline collagen slurry can be more stable to the addition of bioactive glass.

In some aspects, instead of adding the pH adjuster to the slurry, the collagen itself can have its pH adjusted prior to making the slurry. For example, collagen can be immersed in an acid or alkaline bath and agitated for a given period of time in order to adjust the pH. In some aspects, the collagen is then rinsed with water to remove excess salts from the previous pH adjusting bath.

In some aspects, preparing the collagen slurry comprises homogenization. In some aspects, homogenizing can be performed while keeping the temperature below 20° C. results in more uniformity. In some aspects, an ice bath, cooling blanket, or temperature controlled bowl can be used to keep temperatures low. In some aspects, the homogenized slurry can be degassed, for example, via centrifugation or vacuum method. In some aspects, the homogenized slurry can be gently mixed following degassing since gravitational separation can occur.

In some aspects, the collagen slurry can be prepared in a biocompatible buffer. For example, the biocompatible buffer can comprise citrate, acetate, phosphate, formate, tris, MES, and/or succinate buffer systems. The slurry can also comprise additional acids, bases, or salts for pH or handling adjustment, such as sodium hydroxide, potassium hydroxide, hydrochloric acid, acetic acid, sodium carbonate, sodium bicarbonate, sodium sulfate, sulfuric acid, sodium chloride, potassium chloride, sodium acetate, potassium sulfate, potassium carbonate, or potassium bicarbonate.

ii. Adding Bioactive Glass

The disclosed methods of making a composition that is a flowable material can comprise the step of adding bioactive glass to the cross-linked collagen sample.

In some aspects, adding the bioactive glass can be performed before or after the step of lyophilization.

In some aspects, adding the bioactive glass can be performed before or after the step of cross-linking.

In some aspects, bioactive glass can be added dry or pre-mixed with a liquid, such as water or a buffer. In some aspects, 0.3-10 g of bioactive glass powder is added per sheet (or per collagen sample).

In some aspects, adding bioactive glass to the collagen slurry comprises the bioactive glass directly to the collagen slurry.

In some aspects, the bioactive glass can be any of those disclosed herein. In some aspects, the bioactive glass is 45S5, 58S, S53P4, or a Borate-formulation. In some aspects, the bioactive glass comprises 12-45% CaO, 0-65% SiO2, 0-30% Na₂O, 2-17% P₂O₅, 0-7% MgO, 0-1% CaF₂, 0-60% B₂O₃, and 0-15% K₂O by mass percent.

In some aspects, after adding the bioactive glass, the collagen slurry plus bioactive glass mixture is loaded into a delivery device.

In some aspects, the bioactive glass has a particle size of about 10-1000 μm. For example, the composition can have a mean particle size of about 50. In some aspects, the bioactive glass is in the form of particles having a particle size of about 1-600 μm.

In some aspects, the disclosed composition comprise a non-porous bioactive glass. In some aspects, the disclosed composition comprise a porous bioactive glass. In some aspects, the bioactive glass has a porosity of up to 90%. In some aspects, the bioactive glass has pores ranging from about 1 to about 1000 microns. In some aspects, the bioactive glass has an average pore size of 100 microns plus or minus 50 microns, 200 microns plus or minus 50 microns, 300 microns plus or minus 50 microns, 400 microns plus or minus 50 microns, 500 microns plus or minus 50 microns, 600 microns plus or minus 50 microns, 700 microns plus or minus 50 microns.

In some aspects, the bioactive glass is in the form of irregular granules. In some aspects, the bioactive glass is in the form of approximately spherical particles. IN some aspects, the bioactive glass is in the form of fibers

iii. Lyophilization

The disclosed methods of making a composition that is a flowable material can comprise a lyophilization step. In some aspects, lyophilizing comprises lyophilizing the collagen slurry and bioactive glass mixture. In some aspects, the lyophilization can occur in the device that will be used to deliver the flowable material.

In some aspects, the mixture can be frozen. In some aspects, freezing the collagen slurry mixture can be performed at a temperature range of −80° C. to −4° C. In some aspects, freezing the collagen slurry mixture can be performed at a temperature range of −70° C. to −4° C. In some aspects, freezing the collagen slurry mixture can be performed at a temperature range of −50° C. to −4° C.

In some aspects, after freezing, the collagen is then dried. In some aspects, the drying can be performed at the same temperature as the freezing or can be performed at a temperature 1°, 5°, 10°, 15°, 20°, 25°, 30°, 35°, 40°, 45°, or 50° C. higher than the temperature of the freezing step.

In some aspects, the drying step after freezing can be carried out for less than 10 hours. In some aspects, the drying step can be carried out for 10, 12, 14, 16, 18, 20, or 24 hours. In some aspects, the drying step can be carried out for 24, 48 or 72 hours. In some aspects, the drying step can be carried out for 12-144 hours.

iv. First Cross-Linking

In some aspects, the disclosed methods of making a composition that is a flowable material can include the step of cross-linking.

The disclosed methods of making a composition that is a flowable material can comprise the step of crosslinking the collagen slurry to form a cross-linked collagen sample.

In some aspects, the cross-linking can be performed prior to or after lyophilization.

In some aspects, the cross-linking can be performed prior to or after adding the bioactive glass.

In some aspects, a crosslinking agent is mixed in, or added to, the collagen slurry.

In some aspects, the cross-linking agent can be any of those disclosed herein. In some aspects, the cross-linking agent used during the cross-linking step can be, but is not limited to, citric acid, glycine, lysine, glutamic acid, serine, trilysine, glutaraldehyde, transglutaminase, ethylene oxide, phosphate (cross linking aid), tris, carbodiimide, or EDC-NHS. In some aspects, the cross-linking agent is citric acid, wherein the citric acid is in a final concentration of about 1-3%/wt. In some aspects, the cross-linking agent is citric acid, wherein the citric acid is in a final concentration of about 2%/wt.

In some aspects, the second cross-linking step can occur using methods, such as, but not limited to, UV, ethylene oxide, glutaraldehyde vapor, enzymatic crosslinking, dehydrothermal treatment, gamma irradiation, physicochemical crosslinking, and physicothermal crosslinking. Any known method of cross-linking can be used in the disclosed methods.

In some aspects, cross-linking agent concentrations can range from about 0.05% to 5% (on a dry weight basis) of the final composition.

v. Sterilizing

In some aspects, the disclosed methods of making a composition that is a flowable material further comprise the step of sterilizing the compositions. In some aspects, sterilizing can be performed as the final step of the method of producing the disclosed flowable compositions. Thus, in some aspects, sterilizing can be performed after the lyophilization or after cross-linking.

In some aspects, any method of sterilization can be performed, for example, but not limited to, ethylene oxide, gamma irradiation, or ebeam irradiation.

In some aspects, the sterilization occurs with the flowable material inside the delivery device. In some aspects, the sterilization occurs before the flowable material is loaded into the delivery device.

vi. Loading Delivery Device

In some aspects, the disclosed flowable compositions can be loaded into a delivery device. In some aspects, a delivery device can be a syringe or the NovaBone MIS cartridge delivery system as described in U.S. Pat. No. 9,199,032. In some aspects, the delivery device can be any known delivery device.

### F. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of the disclosed compositions.

In some aspects, the disclosed kits can comprise a preloaded cartridge comprising a disclosed composition. In some aspects, the kits can further comprise a suitable adapter to facilitate hydration at time of use, and/or a dispenser to transfer the composition from the cartridge to the treatment site.

In some aspects, the disclosed kits can comprise a preloaded syringe of one of the disclosed lyophilized compositions, an adapter to facilitate hydration, and an additional reservoir to supply the hydration fluid at point of use.

The disclosed kits can also include instructions for how to produce one or more of the disclosed compositions.

### EXAMPLES

### A. Example 1

An example of a disclosed device is an advanced wound care device comprised of bovine collagen and/or bioactive glass. The device can exist in the form of an onlay/overlay where a sheet-like configuration of the product is applied to a wound bed, either pre-hydrated or dry. Alternatively, the device can exist in the form of a flowable material for use in difficult to access wound sites or tunneled wounds. In this configuration, the loose material is packaged in a syringe or other delivery device and is hydrated upon use by mixing of product and hydration fluid between two syringes for a given number of cycles or until other requirements are met.

The device can be used for the management of wounds, including partial wounds, full-thickness wounds, pressure ulcers, venous ulcers, diabetic ulcers, chronic vascular ulcers, tunneled/undermined wounds, surgical wounds, draining wounds, partial thickness burns (2nd degree burns).

### 1. Use of Onlay/Overlay Configuration:

Upon removal from the sterile packaging, the product/device can be applied to the wound dry or placed in a bowl of sterile water or saline to hydrate prior to application. The composition can be allowed to hydrate for an appropriate length of time (e.g., minutes, hours), and the user can gently manipulate product to accelerate hydration. The wound bed can be prepared by removing debris and necrotic tissue and/or surgical debridement. The dry or hydrated product can be cut to a size slightly larger than the wound area and gently applied. Multiple products can be used where adjoined dressings are overlapped to ensure coverage. The disclosed compositions can be secured using an appropriate, non-adherent secondary dressing to maintain device placement and to protect the wound area.

### 2. Use of Flowable Configuration:

Upon removal from the sterile packaging, 3cc of fluid can be added to an empty syringe. This amount is not universal and an appropriate volume of fluid for the given device size is used, for example 3cc for a 5cc device. Using a luer lock connector, the device syringe can be connected to a syringe containing hydration fluid. The hydration fluid can be mixed by mixing the device back and forth between syringes for a total of 10 to 15 mixing cycles; 1 mixing cycle is one complete back and forth motion between the two syringes. The empty syringe can be disconnected and the luer lock connector removed. Alternatively, the device may be contained in a tube for delivery, in which case two syringes are used for hydration. In this configuration, the hydration fluid is loaded in one syringe and connected to one end of the delivery device, while an empty syringe is attached to the opposite end of the delivery device. The fluid is introduced into the delivery device and passed back and forth as needed to hydrate the entire device volume. Immediately after preparing the wound bed by removing debris and necrotic tissue and/or surgical debridement, extrude the hydrated device into the wound site. After application, use an appropriate, non-adherent secondary dressing to maintain device adherence and to protect the wound area.

### 3. Device Sizes

Onlay/overlay configuration: 2 cm×2 cm; 3 cm×3 cm; 5 cm×5 cm; 7.5 cm×7.5 cm; 10 cm×12 cm; 10 cm×25 cm; 17.5 cm×17.5 cm, 20 cm×30 cm

Sheet configurations ranging from 0.5 in² to 100 in², including circular, square, and rectangular sheets.

Flowable configuration: 1 cc; 2.5 cc; 5 cc

### 4. Standard Onlay/Overlay Wound Matrix Product (Monolayer, Collagen/Chondroitin Sulfate)

A 0.75%-2% (m/v) slurry is created using bovine tendon collagen or bovine hide collagen. Porcine or equine-derived collagen can also be used. A higher concentration can be used which will create a denser end product. Increased density will lead to slower resorption of entire product. Depending on the class of product needed (low pH vs high pH), collagen is either acid buffer treated or alkaline buffer treated. Raw collagen is immersed in acid or alkaline bath and agitated for a given period of time [Acid: HCl (can use other acids); Alkaline: NaOH (other alkalines that would be suitable for use include, but are not limited to, KOH, sodium carbonate, sodium phosphate, potassium carbonate, ammonium hydroxide, calcium hydroxide). The collagen is then rinsed with DI water to remove excess salts from previous bath. The collagen is then lyophilized and then milled to appropriate size. An alternative approach is to adjust pH of slurry directly which involves the use of HCl, citric acid, acetic acid for low pH or the use of NaOH or ammonia hydroxide for high pH.

A crosslinking promoter is then added to the slurry. Citric acid is used as crosslinking agent to improve handling of product for low pH product ((0.042 μm per 1 μm collagen, need to convert to mM concentration). Citric acid only improves handling if used in conjunction with dehydrothermal treatment (DHT). Other promoters that can be used are Glycine 1 and 2 mM (used in high pH class of product), Lysine, Glutamic acid, Serine, and Trilysine.

Yeast-derived (or animal derived) chondroitin sulfate is added to the slurry at 0.03 μm per 1 μm collagen. The chondroitin sulfate can be more or less depending on design specifications. The chondroitin sulfate is added for therapeutic effects.

The slurry is homogenized. Homogenizing while keeping temperature below 20° C. results in more uniform sheets. An ice bath is used to keep temperature low. One can also use cooling blanket, temperature controlled bowl, etc. If the temperature is not controlled, homogenization will heat the slurry and result in denaturation of the collagen if the slurry pH is too high or too low.

The homogenized slurry is then degassed which can be degassed via centrifugation. The slurry has to be gently mixed following degassing since phase separation occurs. A preferred method is degassing using vacuum method.

The slurry is then dispensed into molds and lyophilized. The result is then subjected to DHT to produce the final product.

The product is then sterilized via gamma, e-beam, or ethylene oxide.

5. Onlay/Overlay Wound Matrix Product with Bioglass

The above described product can also contain bioactive glass. As such, described is a product with the addition of Bioglass particles. As used herein, the term “Bioglass” or “Bioglass Particles” can be a specific example of a bioactive glass used to produce many of the products in the disclosed Examples.

Bioglass particles are added to slurry following homogenization. Bioglass particles can be formed via melt or sol-gel glass processing methods. Sol-gel derived glass can have dense or porous morphologies. Porous morphology results in higher surface area, leading to faster rates of dissolution.

Bioglass particles can be of various formulations. Examples of bioglass particles are: 45S5, 58S, S53P4, or a Borate-formulation, etc.

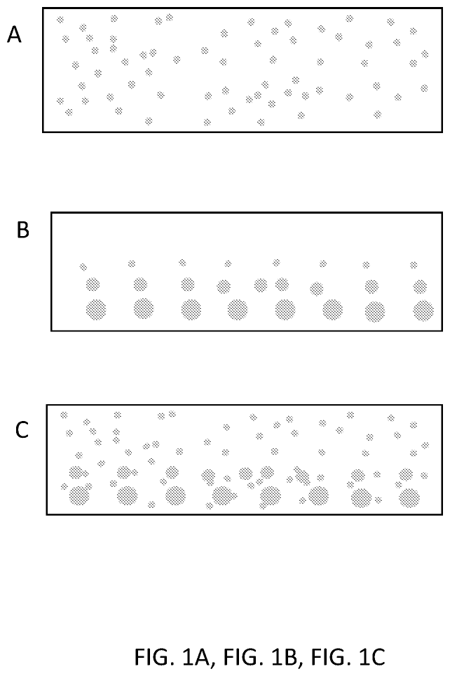

Bioglass particles of various sizes can be added to the collagen slurry. Smaller size range can be used for a homogenously dispersed monolayer product (FIG. 1A). The particles are neutrally buoyant and therefore no settling occurs via gravity. Larger size range can be used for a product that is functionally graded through-the-thickness (FIG. 1B). Larger particles will settle near bottom of mold after slurry is poured and before product is frozen in the lyo process. Settling is caused by gravity and can be accelerated using a vibratory plate. The side of product with the large particles can be the side in direct contact with the wound bed, or can be the side opposite that which contacts the wound bed. A combination of smaller and larger size ranges can be used to create a product with smaller particles dispersed throughout product and large particles that are pre-dominantly on one side (FIG. 1C).

Various crosslinking promoters can be used as in Standard Wound Matrix product. The product undergoes lyophilization and DHT. Further ionic crosslinking can occur via release of bioglass ions in the slurry. The product is then sterilized via gamma, e-beam, or ethylene oxide.

### 6. Onlay/Overlay Bilayer Wound Matrix Product

The onlay/overlay bilayer wound matrix product can be either the standard product or wound matrix with bioglass product (both described above) with the addition of a thin, dense layer of collagen deposited on one side.

The bilayer is for additional functionality. If the bilayer is added on the side that contacts the wound bed it provides an additional denser layer of collagen material to draw MMPs/collagenases away from the wound bed. The bilayer can facilitate the initial wound healing process. If the bilayer is on the opposite side of that which contacts the wound bed it provides a means to slow evaporation of wound fluids since the bilayer is denser. The bilayer can be used in wounds that need to stay moist. The bilayer provides additional means to suture the product in-place.

Taking the above product after lyophilization, a low collagen concentration slurry is spread, painted, or sprayed on the top or bottom surface of the product. The slurry is then air dried or lyophilized to create a thin, dense film of collagen.

The slurry can also include small bioglass particles. The bioglass particles can be formed via melt or sol-gel glass processing methods. The sol-gel derived glass can have dense or porous morphologies. The porous morphology results in higher surface area, leading to faster rates of dissolution. The bioglass particles can be of various formulations. For example, the bioglass particles can be 45S5, 58S, S53P4, or a Borate-formulation, etc.

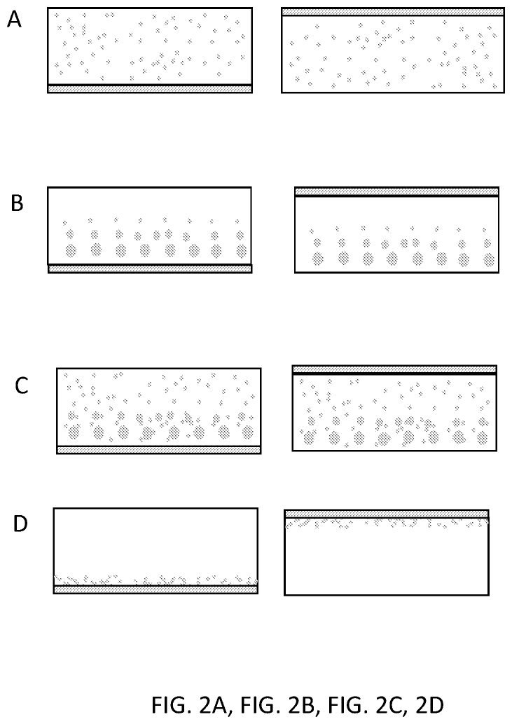

FIG. 2A shows the bilayer on the top or bottom of a product comprising smaller size range of bioglass particles that are homogenously dispersed. FIG. 2B shows the bilayer on the top or bottom of a product comprising comprising larger size range of bioglass particles graded through-the-thickness. FIG. 2C shows the bilayer on the top or bottom of a product comprising a combination of smaller and larger size ranges of bioglass particles with smaller particles dispersed throughout the product and large particles are pre-dominantly on one side. FIG. 2D shows the bilayer on the top or bottom of a product comprising bioglass localized at the interface of the two collagen layers. The product is then sterilized via gamma, e-beam, or ethylene oxide.

### 7. Flowable Wound Matrix-Standard

A 0.75%-2% (m/v) slurry can be created using bovine tendon collagen or bovine hide collagen. Porcine or equine-derived collagen can also be used. Depending on the class of product needed (low pH vs high pH), collagen can either acid buffer treated alkaline buffer treated. Raw collagen can be immersed in acid or alkaline bath and agitated for a given period of time. The acid can be HCl (can use other acids). The alkaline can be NaOH. The collagen is then rinsed with DI water to remove excess salts from the previous bath. The collagen is lyophilized and then milled to an appropriate size. An alternative approach is to adjust the pH of the slurry directly. HCl, citric acid, acetic acid for low pH can be used. NaOH or ammonia hydroxide can be used for higher pH.

A crosslinking promoter is added to the slurry. Citric acid can be used as a crosslinking agent to improve handling of product for low pH product (0.042 μm per 1 μm collagen, need to convert to mM concentration). Other promoters can be used such as glycine 1 and 2 mM (used in high pH class of product), lysine, glutamic acid, serine, or trilysine.

The slurry is homogenized. No temperature control is used. And then the slurry is dispensed into molds. The slurry is then lyophilized. Sheets are milled to <10 mm.

Syringes are filled with appropriate volume/mass of milled collagen and then packaged and sterilized via gamma or e-beam.

8. Flowable Wound Matrix—with Bioglass

The same process as described above for the composition described above up until the slurry is homogenized.

After homogenization, up to 5% (m/v) bioglass is carefully stirred into slurry. The bioglass particles can be formed via melt or sol-gel glass processing methods. Bioglass can have dense or porous morphologies. The porous morphology results in higher surface area, leading to faster rates of dissolution. The bioglass particles can be of various formulations. For example, the bioglass particles can be 45S5, 58S, S53P4, or a Borate-formulation, etc. Bioglass particles of various sizes can be added.

The slurry can then be dispensed into molds. Then, the slurry is lyophilized. Sheets are then milled to fragments into an appropriate size range. Syringes are filled with appropriate volume/mass of milled collagen/glass. The syringes can then be packaged and then sterilized via gamma or e-beam.

A bioglass free version can also be produced and then bioglass can be added during dispensing into a delivery device.

9. Onlay/Overlay Wound Matrix Product with Bioglass

A 0.9%-1.25% (m/v) slurry is created using bovine tendon collagen. Porcine or equine-derived collagen can also be used. A higher concentration can be used which can create a denser end product. Increased density can lead to slower resorption of the entire product. Increased density can make processing more difficult. The collagen is acid buffer treated (as received from supplier). The slurry is prepared by dissolving citric acid (0.03% w/v) and NaOH (8 mM) in cold type I water. The citric acid promotes crosslinking and the NaOH slightly raises pH to improve slurry handling. Hydrating collagen can be in a citric acid/sodium hydroxide solution. Homogenizing can be at max speed for 1-4 min.

A crosslinking promoter is added to the slurry. Citric acid is used as a crosslinking agent to improve handling of product for low pH product (0.03 g per 1 g collagen). Citric acid only improves handling if used in conjunction with DHT. Other promoters can be used such as glycine 1 and 2 mM (used in high pH class of product), lysine, glutamic acid, serine, or trilysine

The slurry is homogenized. Homogenizing performed while keeping temperature below 20° C. results in more uniform sheets. An ice bath, or pre-chilled water, can be used to keep temperature low. A cooling blanket or temperature controlled bowl can also be used. If the temperature is not controlled, homogenization can heat the slurry and result in denaturation of the collagen if the slurry pH is too high or too low.

The slurry is dispensed into lyophilization trays-about IL slurry per tray (can vary between 600-3000 mL to adjust thickness). Lyophilizing the slurry (11.5 in x 23.5 in lyo trays) occurs to make one sheet of lyophilized sponge per shelf. Lyophilized sheets are subjected to DHT to form a mechanically stable intermediate part.

The surface of the intermediate part sheet is perforated (partial thickness) with a pinned roller tool.

0.3-10 g bioglass powder can be added per sheet. The bioglass is dispensed onto the perforated surface and can be spread for even coverage.

An appropriate volume (for example, 600 mL) of type I water is added to the lyophilization tray. The intermediate part sheet is placed with the bioglass side up in the tray to hydrate for at least 5 min.

An additional slurry is prepared as above (similar composition-though can use lower/higher concentrations for this step). An appropriate volume (for example 230 mL) of slurry is poured on top of the hydrated sheet and promptly spread to cover the entire surface with a spatula.

The entire assembly (intermediate part sheet, bioglass, top slurry layer) is lyophilized.

In some aspects, the device sheet can undergo DHT treatment.

The device sheet can be cut to the desired size.

The dry device sheet can be packaged in an appropriate sterile barrier system.

The final product can be sterilized by ethylene oxide, gamma irradiation, or ebeam irradiation.

10. Flowable Wound Matrix Product-MIS Configuration with Bioglass

Collagen from an appropriately qualified source (skin, tendon, or other tissue from bovine, porcine, equine, or other animal type) is lyophilized in the delivery device tube.

Prepare a slurry of collagen (alkaline or acidic) by hydrating collagen and blending it with a homogenizer or other equipment.

No crosslinking is required; acid or base can be added to facilitate handling during manufacturing and/or clinical application. An alkaline slurry can be more stable to bioglass addition.

Bioactive glass is added and hand mixed. The bioactive glass can be added dry or pre-mixed with water or a buffer system to facilitate mixing without destabilizing collagen.

A large syringe is loaded with the mixed slurry and appropriate volumes are ejected into delivery devices. Flexible or rigid fittings can be used to connect large syringes to delivery devices. Loaded delivery devices are placed in a single layer in a lyophilizer tray and then lyophilized. Closures are added to the delivery system as needed. The product can be packaged in an appropriate sterile barrier system with any connectors required for clinical use.

The final product can be sterilized by ebeam or gamma irradiation, or ethylene oxide.

### B. Example 2

### 1. Materials and Methods

Described herein are the materials and methods used to produce an example of the disclosed compositions comprising collagen and bioglass.

Lyophilized acidic bovine tendon collagen was obtained from a qualified supplier. Anhydrous citric acid was obtained from Fisher Scientific, and sodium hydroxide pellets were purchased from Avantor. Deionized water was produced in house. Bioactive glass powder (<90 μm) was produced in house by grinding and sieving 45S5 bioglass frit (Ferro).

To prepare slurry, citric acid and sodium hydroxide were added to cold type I water to a final concentration of 0.03% w/v citric acid and 8 mM sodium hydroxide. The solids were completely dissolved before combining with collagen. The collagen, cut into <2 cm pieces, was combined with the citric acid/sodium hydroxide solution in a glass beaker and mixed manually to ensure that all collagen was wet, then allowed to stand for 5 min to ensure saturation of all collagen. Then, the mixture was homogenized using a Silverson L5M-A overhead homogenizer mixer fitted with a stainless steel slotted disintegrating head. The mixture was processed at ~10,000 rpm for 2-5 min until it formed a viscous homogenous slurry.

The slurry was poured into a 30×60 cm anodized aluminum tray and placed in a Millrock lyophilizer for programmed freeze drying; multiple trays may be used to load all shelves the lyophilizer if desired. The programmed steps chilled the slurry to 0° C., then cooled to −7° C. at ~0.3° C./min, held at −7° C. for 10 min, and cooled to −45° C. at ~1° C./min. After an additional hold at −45° C., the lyophilization recipe proceeded through primary and secondary drying steps at 200 mTorr and temperatures ranging from −10° C. to 25° C. After lyophilization, the spongy collagen sheet was subjected to dehydrothermal treatment (1300 min at 105° C. and 150 mTorr) to improve the handling properties by crosslinking the dry collagen.

To integrate the bioactive glass component, the dry collagen sheet was subjected to partial-thickness perforation over the entire surface. Then, a measured portion of bioactive glass was spread over uniformly over the dry sheet. The sheet was returned to the lyophilization tray, where it was rehydrated with sufficient water to saturate it. Finally, freshly made collagen slurry was spread over the top surface to ensure the bioglass powder was entrapped in the final device. The assembled sheet with bioglass was subjected to lyophilization and dehydrothermal treatment again, as described above, to produce a dry spongy sheet with integrated bioglass.

The collagen-bioglass sheet was cut to size and packaged in Tyvek-PET heat-sealed pouches. It was sterilized by gamma irradiation (25 kGy).

### 2. Use

An embodiment of the disclosed compositions referred to herein as “Wound Matrix” was used to treat full thickness skin wounds in domestic pigs. The wound matrix used herein comprises 78%/wt collagen, 20%/wt bioactive glass, and 2%/wt citric acid. On each pig, 12 full thickness wounds, each 2 cm in diameter, were created on the paravertebral skin. The sites were divided into three treatment groups to compare this embodiment with a legally marketed resorbable wound dressing (Endoform® Natural Dermal Template, Aroa Biosciences) and a negative control (no resorbable wound dressing). The resorbable wound dressing was cut to size and placed in the wound site, covered with saline-soaked gauze, and secured with additional dressings as needed. For the negative control, the saline-soaked gauze was placed directly over the wound site without a resorbable component. Wounds were evaluated macroscopically at 3 day intervals when the dressings were changed. In addition, animals were euthanized at 14, 28 and 45 day timepoints for necropsy and histopathological evaluation. Table 1 shows a comparison of histopathological results for the Wound Matrix and Endoform® devices, an ovine extracellular matrix (ECM), in a full-thickness dorsal wound healing model. Nine domestic hybrid pigs utilized with time points of 14, 28, and 45 days examined.

TABLE 1
Comparison of NovaBone Wound Matrix to Endoform ® Natural
Dermal Template, an ovine extracellular matrix (ECM),
in a full-thickness dorsal wound healing model.

| | | |
|---|---|---|
| | | Endoform ® Natural |
| | | Dermal Template |
| | | Ovine ECM: |
| | | Collagen (I/III/IV) |
| | Wound Matrix | Fibronectin |
| | Type I Collagen | Elastin |
| Composition | Bioactive glass particulate | Hyaluronan |

Results from full-thickness porcine wound study

| | |
|---|---|
| Wound Bed Healing | Both groups exhibited complete re-epithelization by Day 28 |
| Granulation Tissue | Wound Matrix has greater granulation tissue maturity at |
| | Day 14 and Day 28 |
| Inflammation, | Lower magnitude in Wound Matrix compared to |
| superficial/mid | Endoform ® at Day 14 and Day 28 |
| wound bed | |
| Inflammation, | Minimal magnitude in Wound Matrix, greater magnitudes |
| deep wound bed | in Endoform ® at Day 14 and Day 45 |
| Inflammation, | Lower magnitudes in Wound Matrix, greater magnitudes |
| granulomatous | in Endoform ® (including up to marked at Day 45)_{—} |
| wound bed | |
| Inflammation, | Not Observed at any time in Wound Matrix, Mild-to- |
| lymphoid, nodular | moderate incidence/magnitude in Endoform ® at Day 28 |
| wound bed | and Day 45 |

The Wound Matrix device did not impair wound healing based on macroscopic evaluations. Histopathological examination of the wound sites indicated that inflammation in and around the wound bed resolved faster in the wounds treated with Wound Matrix than in the wounds treated with Endoform, the comparison resorbable wound dressing. There was no histopathological evidence that the Wound Matrix device impaired or delayed healing in this wound model.

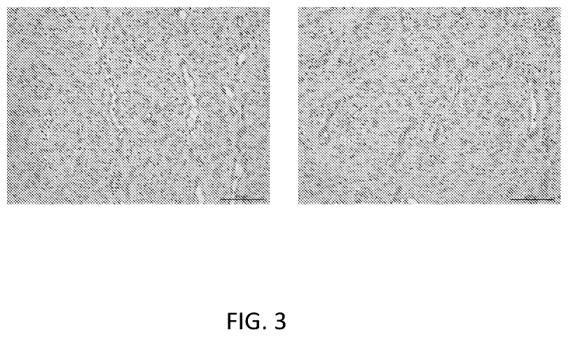

In another study designed to evaluate early healing stages of Wound Matrix in a full-thickness porcine wound healing model, Wound Matrix sites showed consistent neovascularization oriented perpendicular to wound bed (as shown in FIG. 3.). Control sites (saline-soaked gauze, not shown) showed inconsistently oriented capillaries and haphazardly arranged fibroblasts.

The anti-bacterial potential of bioactive glass was studied. The anti-bacterial potential of bioactive glasses has begun to hit a critical mass in the scientific community. Recent studies by Kotsakis, UTHSCSA have shown that fine particulates of 45S5 are effective at inhibiting and even eradicating various strains of bacteria.

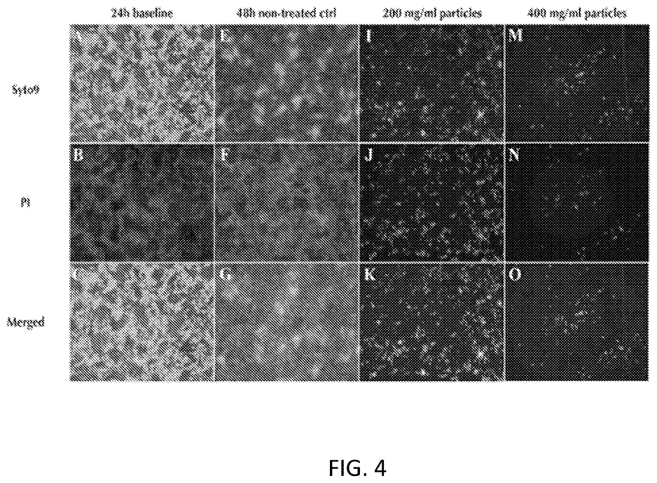

Clinically-derived multi-species biofilm (MRSA, Streptococcus, E. coli, etc.) was almost completely eradicated after 24 hrs exposure to 32-125 μm 45S5 bioactive glass (below left). Cultures of MRSA, in both suspended and biofilm forms, was eradicated at 24 hr. Cultures of P. aeruginosa were eradicated after 48 hr exposure. (see FIG. 4). These results are shown in Table 2.

TABLE 2
anti-bacterial properties of bioactive glass

| | Bacterial | Bacterial |
|---|---|---|
| Species | Reduction, 24 hr | Reduction, 48 hr |
| MRSA, biofilm | 100% | 100% |
| MRSA, nutrient broth | 100% | 100% |
| P. aeruginosa, | 85.6% | 100% |
| nutrient broth | | |

Those skilled in the art will recognize, or be able to ascertain using no more than routine experimentation, many equivalents to the specific embodiments of the method and compositions described herein. Such equivalents are intended to be encompassed by the following claims.

We claim:

1. A composition comprising a) about 5-97%/wt collagen; b) about 0-15%/wt of a cross-linking agent; and c) about 1%-99%/wt bioactive glass.

2. The composition of claim 1, wherein the collagen is marine (e.g. fish), bovine, porcine, ovine, vegan, equine or recombinant collagen.

3. The composition of any of claims 1-2, wherein the collagen is cross-linked.

4. The composition of any of claims 1-3, wherein the cross-linking agent is citric acid, glycine, lysine, glutamic acid, serine, trilysine, glutaraldehyde, transglutaminase, ethylene oxide, phosphate (cross linking aid), tris, carbodiimide, heat, irradiation, or EDC-NHS.

5. The composition of claim 4, wherein the cross-linking agent is citric acid and wherein the citric acid is in a final concentration of about 1-15%/wt.

6. The composition of any one of claims 1-5, wherein the bioactive glass is 45S5, 58S, S53P4, or a Borate-formulation.

7. The composition of any one of claims 1-6, wherein the composition is lyophilized or freeze-dried.

8. The composition of any one of claims 1-7, wherein the composition has a density of about 25+/−20 mg/cc.

9. The composition of any one of claims 1-8, wherein the composition has a pH of about 7-10.

10. The composition of any one of claims 1-9, wherein the composition have a particle size of about 1-1000 μm.

11. The composition of any one of claims 1-11, wherein the bioactive glass has a porosity of up to 90%.

12. The composition of any one of claims 1-11, wherein the bioactive glass has pores ranging from about 1 to about 1000 microns.

13. The composition of any one of claims 1-12, wherein the bioactive glass has an average pore size of 100 microns plus or minus 50 microns.

14. The composition of any one of claims 1-12, wherein the bioactive glass has an average pore size of 200 microns plus or minus 50 microns.

15. The composition of any one of claims 1-12, wherein the bioactive glass has an average pore size of 300 microns plus or minus 50 microns.

16. The composition of any one of claims 1-12, wherein the bioactive glass has an average pore size of 400 microns plus or minus 50 microns.

17. The composition of any one of claims 1-12, wherein the bioactive glass has an average pore size of 500 microns plus or minus 50 microns.

18. The composition of any one of claims 1-12, wherein the bioactive glass has an average pore size of 600 microns plus or minus 50 microns.

19. The composition of any one of claims 1-12, wherein the bioactive glass has an average pore size of 700 microns plus or minus 50 microns.

20. The composition of any one of claims 1-19, wherein the bioactive glass is in the form of particles having a particle size of about 1-600 μm.

21. The composition of any one of claims 1-20, wherein the bioactive glass is in the form of irregular granules.

22. The composition of any one of claims 1-20, wherein the bioactive glass is in the form of approximately spherical particles.

23. The composition of any one of claims 1-20, wherein the bioactive glass is in the form of fibers.

24. The composition of any one of claims 1-23, having a mass of about 0.2-20 grams

25. The composition of any one of claims 1-24, having a volume of about 1-1500 cc.

26. The composition of any one of claims 1-25, having a density of about 25+/−20 mg/cc.

27. The composition of any one of claims 1-26, wherein the composition is sterilized.

28. The composition of any one of claims 1-27, further comprising a layer of collagen deposited on one side of the composition.

29. The composition of any one of claims 1-28, wherein the composition is in a flowable form.

30. The composition of any one of claims 1-29, further comprising a therapeutic agent.

31. The composition of claim 300, wherein the therapeutic agent is a signaling protein, glycosaminoglycan, growth factor, antimicrobial agent, anti-inflammatory agent, analgesic, antibiotic, or a combination thereof.

32. The composition of any one of claims 1-31, wherein the composition does not comprise chondroitin sulfate.

33. A delivery device filled with the composition of any one of claims 29-32.

34. The delivery device of claim 33, wherein the delivery device is a syringe.

35. The delivery device of claim 324, wherein the force required to eject the composition from a syringe applicator is less than 20 lbs.

36. A method for managing a soft tissue defect, comprising: applying the composition of any of claims 1-35 to a soft tissue defect in a patient in need thereof.

37. The method of claim 36, wherein the soft tissue defect is a partial or full thickness wound such as a pressure ulcer, venous ulcer, diabetic ulcer, chronic vascular ulcer, tunneled/undermined wound, surgical wound (donor site/graft, post-Moh's surgery, post-laser surgery, podiatric, wound dehiscence), trauma wound (abrasion, laceration, second-degree burn, or skin tear), or draining wound.

38. A method of making a composition comprising
a) preparing a collagen slurry;
b) lyophilizing the collagen slurry to create a lyophilized collagen sample;
c) crosslinking the lyophilized collagen slurry to form a cross-linked lyophilized collagen sample; and
d) adding bioactive glass to the lyophilized cross-linked collagen sample.

39. The method of claim 38, further comprising rehydrating the cross-linked lyophilized collagen sample to create a rehydrated cross-linked collagen sample;

40. The method of claim 39, further comprising coating the rehydrated cross-linked collagen sample with a collagen slurry.

41. The method of claim 40, further comprising lyophilizing the collagen slurry-coated sample to create a lyophilized collagen-bioglass sample.

42. The method of claim 40 or 41, further comprising cross-linking the collagen-bioglass sample.

43. The method of any one of claims 38-382, wherein the collagen is marine, bovine, porcine, ovine, vegan, equine or recombinant collagen.

44. The method of any one of claims 38-43, further comprising adjusting the pH of the collagen slurry of claim 38 or claim 40.

45. The method of any one of claims 38-44, further comprising a step of milling the lyophilized collagen sample.

46. The method of claim 45, wherein the milling occurs before crosslinking the lyophilized collagen sample.

47. The method of any one of claims 38-46, wherein the crosslinking step comprises contacting the lyophilized collagen slurry with a crosslinking agent.

48. The method of any one of claims 38-46, wherein the crosslinking step comprises UV, ethylene oxide, glutaraldehyde vapor, enzymatic crosslinking, dehydrothermal treatment, physicochemical crosslinking, or physicothermal crosslinking.

49. The method of claim 47, wherein the crosslinking agent is citric acid.

50. The method of claim 49, wherein the composition comprises about 2% citric acid.

51. The method of any one of claims 38-50, wherein adding the bioactive glass comprises perforating the lyophilized collagen sample before adding the bioactive glass.

52. The method of any one of claims 38-49, wherein the collagen slurry coated on the rehydrated collagen sample is the collagen slurry created in step a).

53. The method of any one of claims 38-52, wherein the collagen slurry coated on the rehydrated collagen sample is a different collagen slurry than the collagen slurry created in step a).

54. The method of any one of claims 38-53, wherein the lyophilized collagen sample forms a sponge-like sheet.

55. The method of any one of claims 41-54, further comprising cutting the lyophilized product to a desired size.

56. The method of any one of claims 38-54, wherein the bioactive glass is added to a single side, two sides, or all sides of the rehydrated collagen sample.

57. The method of any one of claims 38-56, wherein the bioglass added in step d) is a single size.

58. The method of any one of claims 38-55, wherein the bioglass added in step d) is of multiple sizes.

59. The method of any one of claims 38-58, wherein the bioglass is dispersed uniformly throughout the composition.

60. The method of any one of claims 38-58, wherein the bioglass is not dispersed uniformly throughout the composition.

61. The method of any one of claims 40-60, wherein the collagen slurry is coated on the same side of the rehydrated collagen sample that contains the bioactive glass.

62. The method of any one of claims 38-61, wherein the composition is a flowable composition.

63. The method of any one of claims 38-63, further comprising milling/homogenizing the lyophilized product.

64. The method of claim 63, further comprising rehydrating the product 63.

65. The method of claim 64, wherein the composition is a flowable composition.

66. The method of claim 65, further comprising loading the product of claim 64 into a delivery device.

67. A method of making a composition comprising
a) preparing a collagen slurry;
b) combining bioactive glass to the collagen slurry to form a bioactive glass-collagen slurry mix; and
c) lyophilizing the bioactive glass-collagen slurry mix to form a lyophilized bioactive glass-collagen product.

68. The method of claim 67, further comprising crosslinking the lyophilized bioactive glass-collagen product to form a cross-linked lyophilized bioactive glass-collagen product; and

69. The method of any one of claims 67-68, further comprising rehydrating the cross-linked lyophilized bioactive glass-collagen product.

70. The method of claim 66, further comprising loading the rehydrated composition into a delivery device.

71. The method of any one of claims 66-70, wherein the bioactive glass is combined with the collagen slurry while the bioactive glass is dry or after the bioactive glass is pre-mixed with a buffer solution.

72. The method of any one of claims 38-71, further comprising sterilizing the composition.

73. The method of claim 72, wherein sterilizing comprises ethylene oxide, gamma irradiation, or ebeam irradiation.

74. A composition made by the method of any of claims 38-73.
